(19) United States
(12) Patent Application Publication
(10) Pub. No.: US 2026/0237513 A1
(43) Pub. Date: Aug. 13, 2026

(54) SYSTEM AND METHOD FOR MULTIMODAL PREDICTION OF PATIENT OUTCOMES

(71) Applicants: Genentech, Inc., South San Francisco, CA (US); Ventana Medical Systems, Inc., Tucson, AZ (US)

(72) Inventors: Raghavan Venugopal, Fremont, CA (US); Zuo Zhao, Mountain View, CA (US); Liping Zhang, Oro Valley, AZ (US); Nazim Nisar Shaikh, Sunnyvale, CA (US); Cyrus T. Manuel, Sunnyvale, CA (US); Md Abid Hasan, Pleasanton, CA (US); Xingwei WANG, Sunnyvale, CA (US); Kandavel Shanmugam, Oro Valley, AZ (US); Yao Nie, Sunnyvale, CA (US); Auranuch Lorsakul, Santa Clara, CA (US)

(21) Appl. No.: 19/088,626

(22) PCT Filed: Oct. 2, 2023
(86) PCT No.: PCT/US2023/034303

§ 371 Date: Mar. 24, 2025

Related U.S. Application Data
(60) Provisional application No. 63/378,164, filed on Oct. 3, 2022.
(60) Provisional application No. 63/533,572, filed on Aug. 18, 2023.

(30) Foreign Application Priority Data
Apr. 30, 2023 (IN) 202311030916
Apr. 30, 2023 (IN) 202311030917
Jun. 30, 2023 (IN) 202311044011

(51) Int. Cl. G16H 50/30; G16H 10/20; G16H 30/40; G16H 50/20

(52) U.S. Cl. CPC G16H 50/30; G16H 10/20; G16H 30/40; G16H 50/20

(57) ABSTRACT

A method of predicting overall survivability of a patient by a prediction system based on machine learning includes receiving, by the prediction system including a processor and a memory, a plurality of input modalities corresponding to the patient, the input modalities being of different types from one another, generating, by the prediction system, a plurality of intermediate features based on the plurality of input modalities, each input modality of the plurality of input modalities corresponding to one or more features of the plurality of intermediate features, and determining, by the prediction system, a survivability score corresponding to an overall survivability of the patient based on a fusion of the plurality of intermediate features.

### CROSS-REFERENCE TO RELATED APPLICATIONS

This application is a bypass continuation application of International Application No. PCT/US2023/034303, filed on Oct. 2, 2023, which claims priority to and the benefit of U.S. Provisional Application No. 63/378,164 entitled SYSTEM AND METHOD FOR MULTIMODAL PREDICTION OF PATIENT OUTCOMES, filed Oct. 3, 2022; U.S. Provisional Application No. 63/533,572 entitled ATTENTION-BASED MULTIMODAL-FUSION FOR NON-SMALL CELL LUNG CANCER (NSCLC) PATIENT SURVIVAL PREDICTION, filed Aug. 18, 2023; India application No. 202311030916 entitled INTEGRATING MULTIMODAL DATA FOR NON-SMALL CELL LUNG CANCER (NSCLC) PATIENT SURVIVAL PREDICTION, filed Apr. 30, 2023; India application No. 202311030917 entitled FEATURE GENERATION AND SELECTION FOR AN EFFICIENT MULTIMODAL ANALYSIS ON BIOLOGICAL DATA, filed Apr. 30, 2023; and India Application No. 202311044011, entitled INTERPRETABLE FEATURE BASED NETWORK FOR CLASSIFYING CELL-OF-ORIGIN FROM WHOLE SLIDE IMAGES IN DIFFUSE LARGE B-CELL LYMPHOMA PATIENTS, filed on Jun. 30, 2023.

### FIELD

One or more aspects of some embodiments according to the present disclosure relate to a system and method for predicting patient outcomes.

### BACKGROUND

Cancers in their various forms have become one of the leading causes of death worldwide. In particular, lung cancer is one of the most prevalent malignancies and the cause of about 25% of all cancer-related deaths. About 84% of the lung cancers are non-small cell lung cancer (NSCLC), which is a group of lung cancers that behave similarly. Immunotherapy with checkpoint inhibitors, such as anti-PD1 and anti-PD-L1 drugs bring promising clinical outcomes for patients with locally advanced (ad) or metastatic (m) NSCLC. However, the biomarkers currently used in selecting patients who can benefit from the targeted or immunotherapy are inaccurate and have much potential for improvement.

The above information disclosed in this Background section is only for enhancement of understanding of the background and therefore the information discussed in this Background section does not necessarily constitute prior art.

### SUMMARY

Aspects of embodiments of the present disclosure are directed to a multi-modal predictive system that utilizes a deep learning framework for predicting overall survival of patients (e.g., NSCLC patients) from diverse multi-modal data. In some embodiments, the deep learning framework leverages digital pathology, genetic information, clinical data, patient demographic data, as well as a host of other modalities to generate predictions that are more accurate than other predictive methods of the related art.

According to some embodiments of the present disclosure, there is a provided method of predicting overall survivability of a patient by a prediction system based on machine learning, the method including: receiving, by the prediction system including a processor and a memory, a plurality of input modalities corresponding to the patient, the input modalities being of different types from one another; generating, by the prediction system, a plurality of intermediate features based on the plurality of input modalities, each input modality of the plurality of input modalities corresponding to one or more features of the plurality of intermediate features; and determining, by the prediction system, a survivability score corresponding to an overall survivability of the patient based on a fusion of the plurality of intermediate features.

In some embodiments, the plurality of input modalities includes: a first modality including histology hematoxylin and eosin (H&E) image data; a second modality including genetic sequencing data; a third modality including clinical data; a fourth modality including radiology magnetic resonance imaging (MRI) data; and a fifth modality including immunohistochemistry (IHC) image data.

In some embodiments, the histology H&E image data includes a digitized image of a tissue sample of the patient that is stained with hematoxylin and eosin dyes.

In some embodiments, the digitized image includes a plurality of images of different tumor regions of the tissue sample.

In some embodiments, the genetic sequencing data includes mRNA gene expressions extracted from a tumorous tissue of the patient.

In some embodiments, the clinical data includes: an age of the patient; a sex of the patient; a tumor stage of the patient; and a performance status of the patient.

In some embodiments, the IHC image data includes a digitized image of a tissue sample of the patient stained with a PD-L1 bio marker.

In some embodiments, the prediction system includes: a first convolutional neural network configured to receive a first modality of the plurality of input modalities and to generate one or more first intermediate features of the plurality of intermediate features; a first feed forward neural network configured to receive a second modality of the plurality of input modalities and to generate one or more second intermediate features of the plurality of intermediate features; a second feed forward network configured to receive a third modality of the plurality of input modalities and to generate one or more third intermediate features of the plurality of intermediate features; a second convolutional neural network configured to receive a fourth modality of the plurality of input modalities and to generate one or more fourth intermediate features of the plurality of intermediate features; and a third convolutional neural network configured to receive a fifth modality of the plurality of input modalities and to generate one or more fifth intermediate features of the plurality of intermediate features.

In some embodiments, the method further includes: combining the first, second, third, and fourth intermediate features to form the fusion of the plurality of intermediate features.

In some embodiments, the prediction system further includes: a fusion layer neural network configured to receive the fusion of the plurality of intermediate features and to generate the survivability score.

In some embodiments, the method further includes: receiving, by a classifier, an input image from a tissue sample of the patient; and extracting, by the classifier, cell spatial graph data from the input image, the cell spatial graph data including a cell type and location of each cell in the input image.

In some embodiments, the input image includes one of a histology H&E image and an IHC image.

In some embodiments, the extracting the cell spatial graph data includes:

detecting cells within the input image; generating cell classification data for the cells detected within the input image, the cell classification data including the cell type and the location of each cell in the input image; and constructing the cell spatial graph data based on the cell classification data.

In some embodiments, the prediction system further includes: a graph convolutional network configured to receive a sixth modality of the plurality of input modalities and to generate one or more sixth intermediate features of the plurality of intermediate features, wherein the sixth modality includes a cell spatial graph corresponding to a histology hematoxylin and eosin (H&E) image or an immunohistochemistry (IHC) image from a tissue sample of the patient.

In some embodiments, the prediction system includes a multi-modal fusion model configured to correlate the plurality of input modalities to the survivability score.

In some embodiments, the method further includes: transmitting the survivability score to a display device for display to a user.

According to some embodiments of the present disclosure, there is provided a method of predicting overall survivability of a patient by a prediction system based on machine learning, the method including: receiving, by the prediction system, a plurality of input modalities corresponding to the patient, the input modalities including: a first modality including histology hematoxylin and eosin (H&E) image data; a second modality including genetic sequencing data; a third modality including clinical data; a fourth modality including radiology magnetic resonance imaging (MRI) data; and a fifth modality including immunohistochemistry (IHC) image data; generating, by the prediction system, a plurality of intermediate features based on the plurality of input modalities, each input modality of the plurality of input modalities corresponding to one or more features of the plurality of intermediate features; and determining, by the prediction system, a survivability score corresponding to an overall survivability of the patient based on a fusion of the plurality of intermediate features.

In some embodiments, the prediction system includes: a first convolutional neural network configured to receive the first modality and to generate one or more first intermediate features of the plurality of intermediate features; a first feed forward neural network configured to receive the second modality and to generate one or more second intermediate features of the plurality of intermediate features; a second feed forward neural network configured to receive the third modality and to generate one or more third intermediate features of the plurality of intermediate features; a second convolutional neural network configured to receive the fourth modality and to generate one or more fourth intermediate features of the plurality of intermediate features; and a third convolutional neural network configured to receive the fifth modality and to generate one or more fifth intermediate features of the plurality of intermediate features.

In some embodiments, the method further includes: receiving, by a classifier of the prediction system, an input image from a tissue sample of the patient; and extracting, by the classifier, cell spatial graph data from the input image, the cell spatial graph data including a cell type and location of each cell in the input image, wherein the input image includes one of the histology H&E image and the IHC image.

According to some embodiments of the present disclosure, there is provided a prediction system including: a first convolutional neural network configured to receive a histology hematoxylin and eosin (H&E) image and to generate one or more first intermediate features; a first feed forward neural network configured to receive genetic sequencing data and to generate one or more second intermediate features; a second feed forward neural network configured to receive clinical data and to generate one or more third intermediate features; a second convolutional neural network configured to receive radiology magnetic resonance imaging (MRI) data and to generate one or more fourth intermediate features; a fusion circuit configured to combine the first, second, third, and fourth intermediate features to form a fusion of intermediate features via attention-gated tensor fusion; and a fusion layer neural network configured to receive the fusion of intermediate features and to generate a survivability score corresponding to an overall survivability of a patient.

### BRIEF DESCRIPTION OF THE DRAWINGS

Non-limiting and non-exhaustive embodiments according to the present disclosure are described with reference to the following figures, wherein like reference numerals refer to like parts throughout the various views unless otherwise specified.

FIG. 1 is a flow diagram illustrating various operations that may occur in a pathology context or pathology environment, according to some embodiments;

FIG. 2 is a block diagram illustrating the prediction system, according to some embodiments of the present disclosure.

FIG. 3 is a block diagram illustrating the prediction system, which utilizes derivative modalities, according to some embodiments of the present disclosure.

FIG. 4 is a block diagram illustrating an internal architecture of the prediction system, according to some embodiments of the present disclosure.

FIG. 5 is a flow diagram illustrating a process of predicting overall survivability of a patient by a prediction system based on machine learning, according to some embodiments of the present disclosure.

### DETAILED DESCRIPTION

Hereinafter, aspects of some example embodiments will be described in more detail with reference to the accompanying drawings, in which like reference numbers refer to like elements throughout. The present invention, however, may be embodied in various different forms, and should not be construed as being limited to only the illustrated embodiments herein. Rather, these embodiments are provided as examples so that this disclosure will be thorough and complete, and will fully convey the aspects and features of the present invention to those skilled in the art. Accordingly, processes, elements, and techniques that are not necessary to those having ordinary skill in the art for a complete understanding of the aspects and features of the present invention may not be described. Unless otherwise noted, like reference numerals denote like elements throughout the attached drawings and the written description, and thus, descriptions thereof will not be repeated. In the drawings, the relative sizes of elements, layers, and regions may be exaggerated for clarity.

Pathology is the medical discipline that attempts to facilitate the diagnosis and treatment of diseases by studying tissue, cell, and fluid samples of patients. In many applications, tissue samples may be collected from patients, and processed into a form that can be analyzed by physicians (e.g., pathologists), often under magnification, by physicians to diagnose and characterize relevant medical conditions based on the tissue sample.

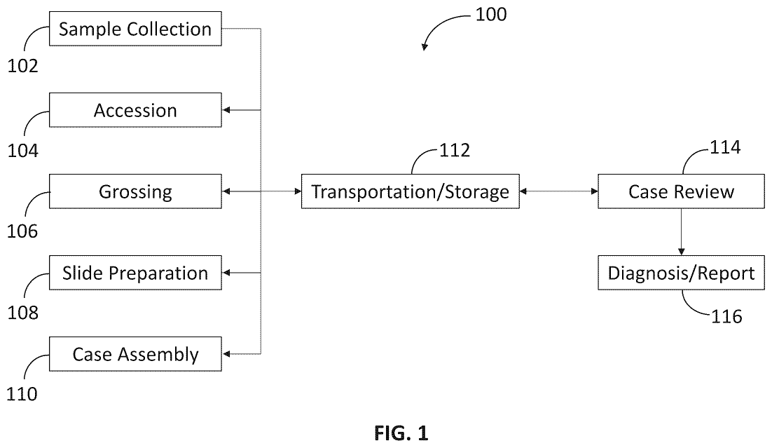

FIG. 1 is a flow diagram illustrating various operations that may occur in a pathology environment or pathology system 100. For example, when a treating physician or medical provider identifies a patient for whom an analysis of a tissue or fluid sample may be beneficial for diagnosing or treating a medical condition, a tissue or fluid sample may be collected at operation 102. The patient's identity may be collected and matched with the patient's sample, and the sample may be placed in a sterile container and/or collection medium for further processing.

The sample may then be transported to a pathology accessioning laboratory at operating 104, where the sample may be received, sorted, organized, and labeled along with other samples from other patients, for further processing.

At operation 106, the sample may be further processed as part of a grossing operation. For example, an individual tissue sample or specimen may be sliced into smaller sections for embedding and subsequent cutting for assembly onto slides.

Then, at operation 108, the sample or specimen may be mounted or deposited on one or more glass slides. The preparation of slides may involve applying one or more reagents or stains to the sample, for example, in order to improve the visibility of, or contrast between, different parts of the sample.

In some instances, at operation 110, several slides, either during the reagent or staining processing, or after the processing is completed, may be assembled or collected in a case or folio. The case may, for example, be carefully labeled with the individual patient's identifying information.

Between each of the operations 102 and 110, at operation 112, the sample, specimen, slide(s), may be transported within the medical facility, or between medical facilities (e.g., between a physician's office and a laboratory), or may be stored between processing operations.

Once the processing of the samples and slides is completed, and a pathologist is ready to review the sample, the slides and/or the case(s) holding multiple slides corresponding to the patient may again be transported, at operation 112, to the pathologist. At operation 114, the pathologist may review the slides, for example, under magnification using a microscope. An individual slide may be placed under the objective lens of the microscope, and the microscope and the slide may be manipulated and adjusted as the pathologist reviews the tissue or fluid.

Once the pathologist has completed the review of the slide, the pathologist may attempt, at operation 116, to form a medical opinion or diagnosis. Meanwhile, the sample or slides may once again be transported, at operation 112, to a longer term storage facility. In some instances, the sample or slides may be again transported, either before or after some storage period, to other physicians for further analysis, second opinions, and the like.

One example of the above-outlined operations may be performed in a pathology environment in which a pathologist identifies patients (e.g., breast cancer patients) who would likely respond to a particular immuno-treatment by analyzing certain information and arriving at a score (e.g., a survivability score) indicative of the treatment's success. Some of the scoring solutions of the related art are single modality systems that use a single bio marker (e.g., PD-L1) or oligo-bio markers. However, often, a single bio marker is not an accurate predictor of the effectiveness of the immunotherapy. For example, in the case of the PD-L1 bio marker, some patients who are negative may respond to treatment, while some who are positive may not. Further, some treatments do not yet have a discovered bio marker that could act as a predictor of the treatment's efficacy.

Accordingly, some aspects of the present disclosure are directed to a deep fusion, multi-modality prediction system that can consider a plethora of disparate data about a patient, such as demographic information, histology of the patient's tumor, the bio marker-stained slides, the stage of illness (e.g., cancer stage), patient's performance data, etc. to obtain an individualized/personalized patient score based on patient's profile. The score may determine how well a patient will respond to a particular treatment.

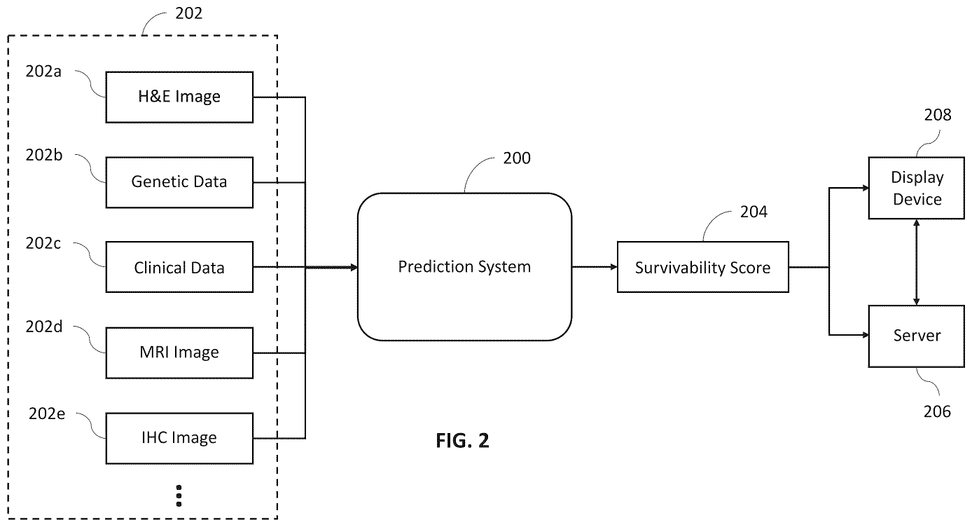

FIG. 2 is a block diagram illustrating the prediction system 200, according to some embodiments of the present disclosure.

According to some embodiments, the prediction system 200 is a multi-modality deep-learning framework that integrates various modality data to predict patient outcomes, and does so in a manner that is more robust than qualitative clinical assessment or unimodal strategies of the related art. In some embodiments, the prediction system 200 is configured to receive a plurality of input modalities 202 that are associated with a patient and to determine a survivability score for the patient based on the modalities 202. The variety of modalities that are utilized by the prediction system 200 may be of different types.

For example, a first modality 202a may include histology hematoxylin and eosin (H&E) image data. The H&E data 202a may include one or more digitized images of a tissue sample (e.g., a tumorous tissue sample) of the patient that is stained with hematoxylin and eosin dyes. H&E dyes stain cell nuclei, extracellular matrix and cytoplasm, and other cell structures, with different colors thus allowing a pathologist and the prediction system 200 to differentiate between different cellular structure. Also, the overall patterns of coloration from the stain show the general layout and distribution of cells and provide a view of a tissue sample's structure. In some examples, the H&E image data 202a may include a plurality of image patches (e.g., three image patches) that are extracted from (e.g., randomly selected and extracted from) a viable tumor region of a stained tissue sample.

The second modality 202b may include genetic sequencing data, such as DNA information and/or mRNA gene expressions of tumor mutation that are extracted from a tumorous tissue of the patient. Each tumor cell may have hundreds or thousands of tumor mutation genes. The second modality 202b may include some or all of the genetic mutations discovered in a tissue sample. In some example, only those expressions that are most relevant to patient survivability may be included in the second modality 202b.

The third modality 202c may include clinical data associated with the patient, such as age, sex, tumor stage, and performance status of the patient. The performance status may be measured by an ECOG score, which describes a patient's level of functioning in terms of the patient's ability to care for oneself, daily activity, and physical ability (e.g., walking, working, etc.). The performance status may also be a Karnofsky performance status, which measures the ability of cancer patients to perform ordinary tasks. The score may range from 0 to 100, with a higher score indicating that the patient is better able to carry out daily activities.

The fourth modality 202d may include radiology magnetic resonance imaging (MRI) data, which may include one or more digitized MRI images (such as Gd-T1w and T2w-FLAIR scans) of a tumor region of the patient. MRI image data may help to assess a tumor's volume.

The fifth modality 202e may include immunohistochemistry (IHC) image data. The IHC data 202e may include one or more digitized images of a tissue sample (e.g., a tumorous tissue sample) of the patient that is stained with a PD-L1 bio marker. The PD-L1 bio marker may produce brown stains when the antibody is able to attach to those tumor cells that have the PD-L1 expression. The IHC image may correspond to a slice of a tissue sample that is adjacent to the slice that the H&E image 202a is based on. In some examples, the cellular structure captured in the H&E and IHC images may be the same or substantially the same; however, that is not necessarily the case. For example, while a H&E image may provide information about the pattern, shape, and structure of cells in a tissue sample, an IHC image, which shows the distribution and localization of specific proteins in a sample, may not clearly reveal the cellular structure of the tissue sample.

Once the prediction system 200 estimates a survivability score 204, the score may be transmitted to a server (e.g., a remote server or a cloud server) 206 for further processing and/or to a display device 208 for display to a user.

While the description above describes five modalities as examples of the input modalities to the prediction system 200, embodiments of the present disclosure are not limited thereto, and any suitable type of modality and/or any suitable number of modalities may be employed by the prediction system 200 to determine the survivability score 204.

For example, the prediction system 200 may utilize one or more derivates of the plurality of modalities 202.

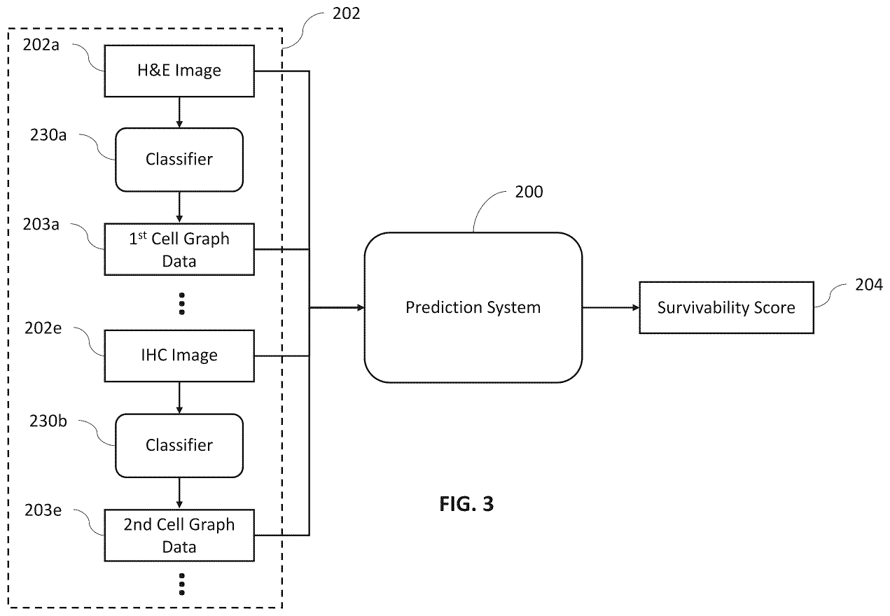

FIG. 3 is a block diagram illustrating the prediction system 200, which utilizes derivative modalities, according to some embodiments of the present disclosure.

According to some embodiments, the cellular structure captured by the H&E image data 202a or the IHC image data 202e is used to generate cell graph data that identifies the location and cell type of each cell within the image. In general, graphs may represent spatial arrangements and neighborhood relationships of different tissue components, which may be characteristics observed visually by pathologists during investigation of specimens. Depending on the adopted approach (e.g., Voronoi Diagram, Delaunay Triangulation, Nearest Neighbor Graph, etc.), the nodes and edges of a graph may represent different elements or characteristics. For example, each node in the graph may identify a cell (e.g., a center of nucleus of a cell), and each edge may identify a Euclidean distance between adjacent cells or represent the similarity between adjacent cells. The prediction system 200 may utilize the cell graph data to extract features that it can use for survival outcome prediction. The cell classification may be performed manually by a pathologist or may be performed by a trained classifier.

In some embodiments, a classifier (e.g., a machine learning classifier) 230a/b receives an input image, which is an image of a stained tissue sample (e.g., an H&E image 202a or an IHC image 202e), detects the cells within the input image, and generates cell classification data corresponding to the detected cells. The classification data, which includes the type and location of each cell in the input image, may be used to generate the cell graph data 203a/e.

In some embodiments, the classifier 230a/b includes a neural network (e.g., a convolutional neural network) capable of cell detection and cell classification. The neural network may include a number of layers each of which performs a convolutional operation, via the application of the kernels/filters, on an input feature map (IFM) to generate an output feature map, which serves as the input feature map of a subsequent layer. In the first layer of the neural network, the input feature map may be the input image (e.g., H&E image 202a or an IHC image 202e). The neural network may be a convolutional neural network (ConvNet/CNN), which can take in an input image, assign importance (e.g., via learnable weights and biases) to various aspects/objects in the image and be able to differentiate one from the other. However, embodiments of the present disclosure are not limited thereto. For example, the neural network may be a recurrent neural network (RNN) with convolution operation, a random forest network, or the like. In some embodiments, the neural network additionally generate the spatial cell graph 203a/e based on the classification data.

As shown in FIG. 3, in some embodiments, a first classifier 230a generates first cell graph data 203a based on the H&E image data 202a, and a second classifier 230b generates second cell graph data 203b based on the IHC image data 202e. However, embodiments of the present invention are not limited thereto. For example, the first and second cell graph data 203a and 203b may be generated by the same classifier 230a/b, as opposed to two separate classifiers. Further, in some embodiments, only one of the first and second cell graph data 203a and 203b may be generated and utilized by the prediction system 200. While the classifiers 230a and 230b are shown as being external to the prediction system 200, embodiments of the present disclosure are not limited thereto, and one or more of the first and second classifiers 230a and 230b may be included in (e.g., be part of) the prediction system 200.

The prediction system may include a multi-modal fusion model (e.g., a multi-modal deep orthogonal fusion (DOF) model) configured to correlate the plurality of input modalities to the survivability score. In some embodiments, the prediction system 200 includes a plurality of neural networks (e.g., a plurality of unimodal neural networks) for processing the multitude of input modalities 202. Each modality may have a corresponding neural network of suitable type.

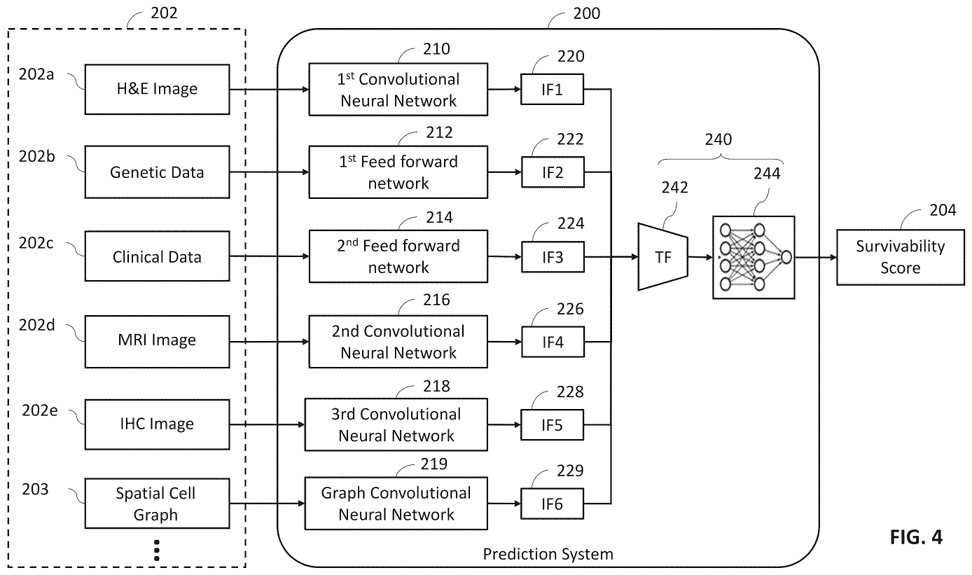

FIG. 4 is a block diagram illustrating an internal architecture of the prediction system 200, according to some embodiments of the present disclosure.

According to some embodiments, the prediction system 200 includes a plurality of neural networks (also referred to as unimodal networks/submodels or feature extraction models) corresponding to (e.g., having a one-to-one correspondence with) the plurality of modalities. Each neural network generates one or more intermediate features (IFs; also referred to as data modality feature representations or modality feature vectors) that correspond to the input modality. The intermediate features from the various neural networks are fused and utilized by the prediction system 200 to generate the survivability score 204. In some examples, the intermediate features are self-identified by the prediction system 200 during training. However, embodiments of the present disclosure are not limited thereto, and one or more of the intermediate features may be manually selected by a user of the prediction system 200 during training of the prediction system 200.

In some embodiments, the prediction system 200 includes a first convolutional neural network (e.g., a U-Net) 210 for processing the H&E image data of the first modality 202a to generate a corresponding one or more first intermediate data (e.g., histology features; IF1) 220; a first feed forward network 212 for processing the genetic data of the second modality 202b to generate a corresponding one or more second intermediate data (e.g., genetic features; IF2) 222; a second feed forward network (or a fully connected (FC) network, etc.) 214 for processing the clinical data of the third modality 202c to generate a corresponding one or more third intermediate data (e.g., clinical features; IF3) 224; a second convolutional neural network 216 for processing the MRI image data of the fourth modality 202d to generate a corresponding one or more fourth intermediate data (e.g., MRI features; IF4) 226; and a third convolutional neural network 218 for processing the IHC image data of the fifth modality 202e to generate a corresponding one or more fifth intermediate data (e.g., IHC features; IF5) 228. In some example, one or more of the input modalities 202 may be combined and fed to the same network. For example, the genetic data 202b and the clinical data 202c may be combined before being provided to a single feed forward network, FC network, or the like.

In some embodiments, the prediction system 200 includes a graph convolutional neural network 219 for processing spatial cell graph data of a sixth modality 203 (e.g., 203a/b), which may correspond to (e.g., be based on or extracted from) H&E image data or IHC image data, to generate a corresponding one or more sixth intermediate data (e.g., graph features; IF6) 229. In embodiments in which cell graph data is extracted from two or more images (e.g., both the H&E image and IHC image), one or more additional graph convolutional networks may be used by the prediction system 200 to generate additional intermediate features.

Thus, each input data modality 202 is handled by a machine learning (e.g., a dedicated deep-learning submodel/unimodel) 210/ . . . /219, which is trained to generate intermediate data (e.g., modality-specific feature representations or feature representation vectors) 220/ . . . /229.

In some embodiments, the prediction system 200 also includes a fusion network 240 that combines the generated feature representation vectors into a single fused representation (e.g., single fused vector), and uses the fused representation vector to output a survivability score (also referred to as a fused prognostic risk score). The fusion network 240 may include a data fusion block (e.g., fusion circuit) 242 and a fusion layer neural network 244. The fusion block 242 is configured to combine (e.g., fuse) the various intermediate features (e.g., IF1-IF6) generated by the plurality of unimodal neural networks (e.g., 210, 212, 214, 216, 218, and 219) via, for example, attention-gated tensor fusion, which in part controls the expressiveness of each modality 202. The fusion layer neural network 244 is configured to generate the survivability score based on the fusion of the plurality of intermediate features by, e.g., utilizing a Cox partial likelihood loss function. The fusion layer neural network 244 may include an integrated model of pairwise feature interactions across modalities. In some examples, the fusion layer neural network 244 may be an FC network (composed of several fully-connected layers), or the like.

The neural networks making up the prediction system 200 may be trained in one shot through one end-to-end training session with a large training data set, or the different neural networks corresponding to the modalities may be separately trained and then combined together to form one large system, i.e., the prediction system 200. For example, in general, the unimodal networks 202 can be first trained separately for survival prediction. However, one or more of the image modality feature extraction models 202a, 202d, and 202e can be a pre-trained model that is trained on a separate public dataset (e.g. ImageNet) for a different task. This may be done to leverage non-medical data to learn richer feature representation. Then, during the multimodal network training phase, the fusion network (which includes the fusion block 242 and the fusion layer neural network 244) is trained using the features extracted by the trained unimodal networks 202 in the first step as the inputs. During multimodal network training, the unimodal networks 202 may be either frozen, unfrozen or frozen in the first few epochs and unfrozen afterwards, to allow for further optimization of the unimodal feature extraction.

The holistic approach of the prediction system 200, which utilizes a multitude of modalities associated with a patient, allows the system 200 to make more precise predictive scores for each individual patient.

Table 1 compares the performance of the prediction system 200 with that of other approaches, based on the concordance index (CI), which is a standard performance measure for model assessment in survival analysis. In Table 1, the baseline represents the statistical technique used to predict overall survival, whereas other models use deep-learning predictions. As illustrated in the example of Table 1, despite the fact that the fusion method of the prediction system 200 uses only three modalities, its predictive capability outperforms the unimodality method of histology H&E and the two-modality method of omics (clinical and mRNA).

TABLE 1

| | Train C-Index | Test C-Index |
|---|---|---|
| Model | (100 samples) | (22 samples) |
| Baseline: Cox (Clinical Data) | 0.60 | 0.50 |
| Histology H&E | 0.53 | 0.44 |
| Omic (Clinical + mRNA) | 0.90 | 0.49 |
| Fusion (H&E + Clinical + mRNA) | 0.98 | 0.54 |

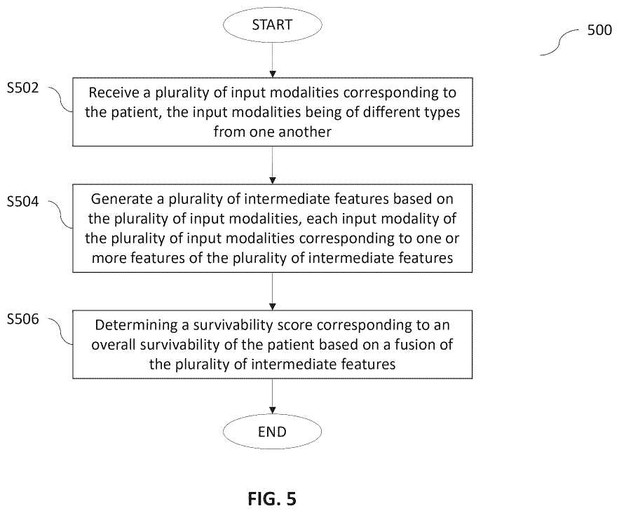

FIG. 5 is a flow diagram illustrating a process 500 of predicting overall survivability of a patient by a prediction system based on machine learning, according to some embodiments of the present disclosure.

According to some embodiments, the prediction system 200 receives a plurality of input modalities 202 corresponding to the patient, the input modalities being of different types from one another (S502). The plurality of input modalities may include one or more of a first modality 202a including histology hematoxylin and eosin (H&E) image data; a second modality 202b including genetic sequencing data; a third modality 202c including clinical data; a fourth modality 202d including radiology magnetic resonance imaging (MRI) data; and a fifth modality 202e including immunohistochemistry (IHC) image data.

In some embodiments, the prediction system 200 then generates a plurality of intermediate features based on the plurality of input modalities, each input modality of the plurality of input modalities corresponding to one or more features of the plurality of intermediate features (S504). The prediction system 200 combines the intermediate features to form a fusion of the intermediate features via attention-gated tensor fusion.

The prediction system 200 determines a survivability score corresponding to an overall survivability of the patient based on the fusion of intermediate features (S506).

As described above, the prediction system, according to some embodiments, fuses heterogeneous multimodal data using one single deep learning framework for patient survival prediction. The prediction system enables better characterization of interactions between different modalities and allows clinicians to gain more insight from the rich clinical and diagnostic information to build an integrated-personalized healthcare solution. Additionally, the prediction system provides a more precise predictive score for each individual patient than single- or oligo-parameters such as demographics, histology, PD-L1 status and omics, etc. The score may be used for selecting the best treatment regimen for that patient.

According to various embodiments of the present disclosure, the prediction system 200 is implemented using one or more processing circuits or electronic circuits configured to perform various operations as described above. Types of electronic circuits may include a central processing unit (CPU), a graphics processing unit (GPU), an artificial intelligence (AI) accelerator (e.g., a vector processor, which may include vector arithmetic logic units configured efficiently perform operations common to neural networks, such dot products and softmax), a field programmable gate array (FPGA), an application specific integrated circuit (ASIC), a digital signal processor (DSP), or the like. For example, in some circumstances, aspects of embodiments of the present disclosure are implemented in program instructions that are stored in a non-volatile computer readable memory where, when executed by the electronic circuit (e.g., a CPU, a GPU, an AI accelerator, or combinations thereof), perform the operations described. The operations performed by the prediction system 200 may be performed by a single electronic circuit (e.g., a single CPU, a single GPU, or the like) or may be allocated between multiple electronic circuits (e.g., multiple GPUs or a CPU in conjunction with a GPU). The multiple electronic circuits may be local to one another (e.g., located on a same die, located within a same package, or located within a same embedded device or computer system) and/or may be remote from one other (e.g., in communication over a network such as a local personal area network such as Bluetooth®, over a local area network such as a local wired and/or wireless network, and/or over wide area network such as the internet, such a case where some operations are performed locally and other operations are performed on a server hosted by a cloud computing service). One or more electronic circuits operating to implement the prediction system 200 may be referred to herein as a computer or a computer system, which may include memory storing instructions that, when executed by the one or more electronic circuits, implement the systems and methods described herein.

The terminology used herein is for the purpose of describing particular embodiments only and is not intended to be limiting of the present invention. As used herein, the singular forms “a” and “an” are intended to include the plural forms as well, unless the context clearly indicates otherwise. It will be further understood that the terms “comprises,” “comprising,” “includes,” and “including,” when used in this specification, specify the presence of the stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list.

It will be understood that, although the terms “first,” “second,” “third,” etc., may be used herein to describe various elements, components, regions, layers and/or sections, these elements, components, regions, layers and/or sections should not be limited by these terms. These terms are used to distinguish one element, component, region, layer or section from another element, component, region, layer or section. Thus, a first element, component, region, layer or section described below could be termed a second element, component, region, layer or section, without departing from the spirit and scope of the present invention.

As used herein, the term “substantially,” “about,” and similar terms are used as terms of approximation and not as terms of degree, and are intended to account for the inherent deviations in measured or calculated values that would be recognized by those of ordinary skill in the art. Further, the use of “may” when describing embodiments of the present invention refers to “one or more embodiments of the present invention.” As used herein, the terms “use,” “using,” and “used” may be considered synonymous with the terms “utilize,” “utilizing,” and “utilized,” respectively.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

Although aspects of some example embodiments of the system and method of quantification of a pathology slide using a cell-based scoring system have been described and illustrated herein, various modifications and variations may be implemented, as would be understood by a person having ordinary skill in the art, without departing from the spirit and scope of embodiments according to the present disclosure. Accordingly, it is to be understood that a pathology slide manufacturing system and method according to the principles of the present disclosure may be embodiment other than as specifically described herein. The disclosure is also defined in the following claims, and equivalents thereof.

What is claimed is:

1. A method of predicting overall survivability of a patient by a prediction system based on machine learning, the method comprising:
receiving, by the prediction system comprising a processor and a memory, a plurality of input modalities corresponding to the patient, the input modalities being of different types from one another;
generating, by the prediction system, a plurality of intermediate features based on the plurality of input modalities, each input modality of the plurality of input modalities corresponding to one or more features of the plurality of intermediate features; and
determining, by the prediction system, a survivability score corresponding to an overall survivability of the patient based on a fusion of the plurality of intermediate features.

2. The method of claim 1, wherein the plurality of input modalities comprises:
a first modality comprising histology hematoxylin and eosin (H&E) image data;
a second modality comprising genetic sequencing data;
a third modality comprising clinical data;
a fourth modality comprising radiology magnetic resonance imaging (MRI) data; and
a fifth modality comprising immunohistochemistry (IHC) image data.

3. The method of claim 2, wherein the histology H&E image data comprises a digitized image of a tissue sample of the patient that is stained with hematoxylin and eosin dyes.

4. The method of claim 3, wherein the digitized image comprises a plurality of images of different tumor regions of the tissue sample.

5. The method of claim 2, wherein the genetic sequencing data comprises mRNA gene expressions extracted from a tumorous tissue of the patient.

6. The method of claim 2, wherein the clinical data comprises:
an age of the patient;
a sex of the patient;
a tumor stage of the patient; and
a performance status of the patient.

7. The method of claim 2, wherein the IHC image data comprises a digitized image of a tissue sample of the patient stained with a PD-L1 bio marker.

8. The method of claim 1, wherein the prediction system comprises:
a first convolutional neural network configured to receive a first modality of the plurality of input modalities and to generate one or more first intermediate features of the plurality of intermediate features;
a first feed forward neural network configured to receive a second modality of the plurality of input modalities and to generate one or more second intermediate features of the plurality of intermediate features;
a second feed forward network configured to receive a third modality of the plurality of input modalities and to generate one or more third intermediate features of the plurality of intermediate features;
a second convolutional neural network configured to receive a fourth modality of the plurality of input modalities and to generate one or more fourth intermediate features of the plurality of intermediate features; and
a third convolutional neural network configured to receive a fifth modality of the plurality of input modalities and to generate one or more fifth intermediate features of the plurality of intermediate features.

9. The method of claim 8, further comprising:
combining the first, second, third, and fourth intermediate features to form the fusion of the plurality of intermediate features.

10. The method of claim 8, wherein the prediction system further comprises:
a fusion layer neural network configured to receive the fusion of the plurality of intermediate features and to generate the survivability score.

11. The method of claim 1, further comprising:
receiving, by a classifier, an input image from a tissue sample of the patient; and
extracting, by the classifier, cell spatial graph data from the input image, the cell spatial graph data comprising a cell type and location of each cell in the input image.

12. The method of claim 11, wherein the input image comprises one of a histology H&E image and an IHC image.

13. The method of claim 11, wherein the extracting the cell spatial graph data comprises:
detecting cells within the input image;
generating cell classification data for the cells detected within the input image, the cell classification data comprising the cell type and the location of each cell in the input image; and
constructing the cell spatial graph data based on the cell classification data.

14. The method of claim 1, wherein the prediction system further comprises:
a graph convolutional network configured to receive a sixth modality of the plurality of input modalities and to generate one or more sixth intermediate features of the plurality of intermediate features,
wherein the sixth modality comprises a cell spatial graph corresponding to a histology hematoxylin and eosin (H&E) image or an immunohistochemistry (IHC) image from a tissue sample of the patient.

15. The method of claim 1, wherein the prediction system comprises a multi-modal fusion model configured to correlate the plurality of input modalities to the survivability score.

16. The method of claim 1, further comprising:
transmitting the survivability score to a display device for display to a user.

17. A method of predicting overall survivability of a patient by a prediction system based on machine learning, the method comprising:
receiving, by the prediction system, a plurality of input modalities corresponding to the patient, the input modalities comprising:
a first modality comprising histology hematoxylin and eosin (H&E) image data;
a second modality comprising genetic sequencing data;
a third modality comprising clinical data;
a fourth modality comprising radiology magnetic resonance imaging (MRI) data; and
a fifth modality comprising immunohistochemistry (IHC) image data;
generating, by the prediction system, a plurality of intermediate features based on the plurality of input modalities, each input modality of the plurality of input modalities corresponding to one or more features of the plurality of intermediate features; and
determining, by the prediction system, a survivability score corresponding to an overall survivability of the patient based on a fusion of the plurality of intermediate features.

18. The method of claim 17, wherein the prediction system comprises:
a first convolutional neural network configured to receive the first modality and to generate one or more first intermediate features of the plurality of intermediate features;
a first feed forward neural network configured to receive the second modality and to generate one or more second intermediate features of the plurality of intermediate features;
a second feed forward neural network configured to receive the third modality and to generate one or more third intermediate features of the plurality of intermediate features;
a second convolutional neural network configured to receive the fourth modality and to generate one or more fourth intermediate features of the plurality of intermediate features; and
a third convolutional neural network configured to receive the fifth modality and to generate one or more fifth intermediate features of the plurality of intermediate features.

19. The method of claim 17, further comprising:
receiving, by a classifier of the prediction system, an input image from a tissue sample of the patient; and
extracting, by the classifier, cell spatial graph data from the input image, the cell spatial graph data comprising a cell type and location of each cell in the input image,
wherein the input image comprises one of the histology H&E image and the IHC image.

20. A prediction system comprising:
a first convolutional neural network configured to receive a histology hematoxylin and eosin (H&E) image and to generate one or more first intermediate features;
a first feed forward neural network configured to receive genetic sequencing data and to generate one or more second intermediate features;
a second feed forward neural network configured to receive clinical data and to generate one or more third intermediate features;
a second convolutional neural network configured to receive radiology magnetic resonance imaging (MRI) data and to generate one or more fourth intermediate features;
a fusion circuit configured to combine the first, second, third, and fourth intermediate features to form a fusion of intermediate features via attention-gated tensor fusion; and
a fusion layer neural network configured to receive the fusion of intermediate features and to generate a survivability score corresponding to an overall survivability of a patient.
